(19) United States
(12) Patent Application Publication
(10) Pub. No.: US 2019/0217076 A1
(43) Pub. Date: Jul. 18, 2019

(54) Systems, Devices, and/or Methods for Managing Medical Treatments

(71) Applicant: Terrence Jay O'Neil, Johnson City, TN (US)

(72) Inventor: Terrence Jay O'Neil, Johnson City, TN (US)

(21) Appl. No.: 16/238,662

(22) Filed: Jan. 3, 2019

Related U.S. Application Data
(60) Provisional application No. 62/618,556, filed on Jan. 17, 2018.

(51) Int. Cl. A61M 39/16; A61M 39/10

(52) U.S. Cl. CPC A61M 39/165; A61M 39/10; A61M 2039/1066; A61M 2039/1016; A61M 2207/00

(57) ABSTRACT

Certain exemplary embodiments can provide a system comprising a shell that comprises an upper portion and a lower portion. The upper portion is coupled to the lower portion via a hinge. The upper portion defines a first upper slot and a second upper slot. The lower portion defines a first lower slot and a second lower slot. The upper portion comprises a clasp. The lower portion defines a clasp receptacle.

### CROSS-REFERENCES TO RELATED APPLICATIONS

This application claims priority to, and incorporates by reference herein in its entirety, pending U.S. Provisional Patent Application Ser. No. 62/618,556 (Attorney Docket No1262-01), filed Jan. 18, 2018.

### BRIEF DESCRIPTION OF THE DRAWINGS

FIGS. 1, 2, 3A, 3B, 3C, 3D, 3E, 3F, 3G, and FIG. 7 are executed in color.

A wide variety of potential practical and useful embodiments will be more readily understood through the following detailed description of certain exemplary embodiments, with reference to the accompanying exemplary drawings in which:

FIG. 1 is a perspective view of an exemplary embodiment of a system 1000;

FIG. 2 is a perspective view of an exemplary embodiment of a system 2000;

FIG. 3A is a perspective view of an exemplary embodiment of a system 3000;

FIG. 3B is a perspective view of an exemplary embodiment of a system 3100;

FIG. 3C is a perspective view of an exemplary embodiment of a system 3200;

FIG. 3D is a perspective view of an exemplary embodiment of a system 3300;

FIG. 3E is a perspective view of an exemplary embodiment of a system 3400;

FIG. 3F is a perspective view of an exemplary embodiment of a system 3500;

FIG. 3G is a perspective view of an exemplary embodiment of a system 3600;

FIG. 4 is a perspective view of an exemplary embodiment of a system 4000; and

FIG. 5 is a perspective view of an exemplary embodiment of a system 5000; and

FIG. 6A is a perspective view of an exemplary embodiment of a system 6000;

FIG. 6B is a perspective view of system 6000;

FIG. 6C is a clip cross section 6100 of system 6000 with the clip open;

FIG. 6D is a clip cross section 6100 of system 6000 with the clip shut; and

FIG. 7 is a perspective view of an exemplary embodiment of a system 7000.

### DETAILED DESCRIPTION

Certain exemplary embodiments can provide a system comprising a shell that comprises an upper portion and a lower portion. The upper portion is coupled to the lower portion via a hinge. The upper portion defines a first upper slot and a second upper slot. The lower portion defines a first lower slot and a second lower slot. The upper portion comprises a clasp. The lower portion defines a clasp receptacle.

Certain exemplary embodiments address issues with infections in patients such as those that undergo dialysis. Certain exemplary embodiments resist entry of bacteria into patients' circulatory systems, thereby reducing a chance of a primary source of infections.

Indwelling vascular catheters are generally colonized by microorganisms within 24 hours after insertion. Bacteria are introduced into the lumen through the flora of the surrounding skin or the hands of health care workers during catheter hub manipulation.

Certain exemplary embodiments reduce changes of bloodstream infections, which presently are leading to a higher-than-desired rate of hospitalizations and deaths in dialysis patients. Certain exemplary embodiments are secured the hubs during use but protected them from contamination during line connection by surrounding and enclosing the catheter hub or dialysis line connection securely. Certain exemplary embodiments reduce risks of catheter hub contamination.

Certain exemplary embodiments provide a bivalved/clamshell device with a securing latch. Such devices engage both sides of a catheter. Certain exemplary embodiments substantially completely encircle/enclose bloodline connections and therefore protects them against contamination.

Certain exemplary embodiments engage both sides of a bloodline connection, thereby reducing the inadvertent disconnection of the bloodlines. Certain exemplary embodiments substantially completely encircle and enclose bloodline connections and thereby protect them against contamination.

Certain exemplary embodiments comprise a securing latch to prevent opening and disconnection due to patient manipulation or a forceful tug on the tubing. Certain exemplary embodiments comprise on a clamshell to permit easy opening and positioning.

Certain exemplary embodiments are substantially transparent thereby permitting positive visualization of the connector status during use.

Certain exemplary embodiments comprise a closure spring to assist in maintaining closure if the closure latch is not properly engaged. Certain exemplary embodiments encircle connections substantially completely and engage both sides of the connection.

Certain exemplary embodiments comprise a closure latch and wings on the clamshell to permit easier opening and positioning. Certain exemplary embodiments engage a separate winged engagement device, thereby rendering the securing of the lines substantially complete.

Certain exemplary embodiments engage with medical fluid lines and reduce risks of catheters becoming dislodged and reducing the inadvertent disconnection of bloodlines. Certain exemplary embodiments substantially encircle and secure the connection of two medical fluid lines. Certain exemplary embodiments encircle and enclose connections point and prevent dislodgement and contamination of the connection.

Certain exemplary embodiments are designed to secure two Luer-locked medical fluid lines. Certain exemplary embodiments prevent inadvertent disconnection of two Luer-locked bloodlines. Certain exemplary embodiments provide a clamshell/bivalved device, which is constructed to encircle and secure two medical fluid lines.

Certain exemplary embodiments are constructed for hemodialysis use and would be useable in that context. Certain exemplary embodiments are constructed for peritoneal dialysis. Certain exemplary embodiments enclose connections in such a way as to positively prevent contamination.

Certain exemplary embodiments are substantially transparent, which permits positive visual confirmation of the status of connections during use. Certain exemplary embodiments provide a bivalved/clamshell-type device.

Certain exemplary embodiments substantially enclose two fluid lines carrying fluid under pressure. Certain exemplary embodiments provide spring closure-assist and wings to assist opening.

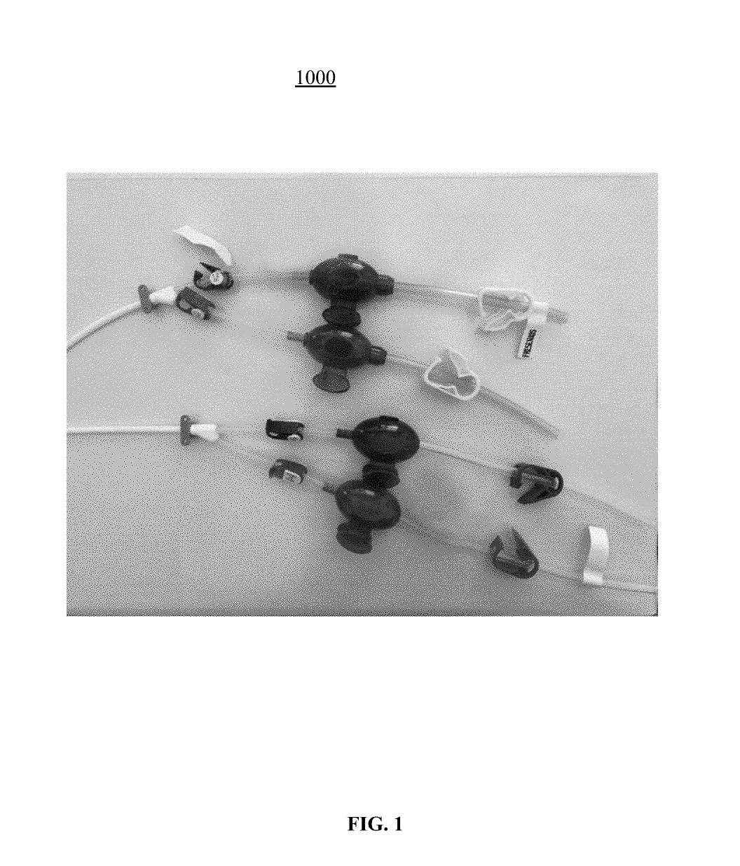

FIG. 1 is a perspective view of an exemplary embodiment of a system 1000, which illustrates exemplary dialysis lines coupled via exemplary couplings.

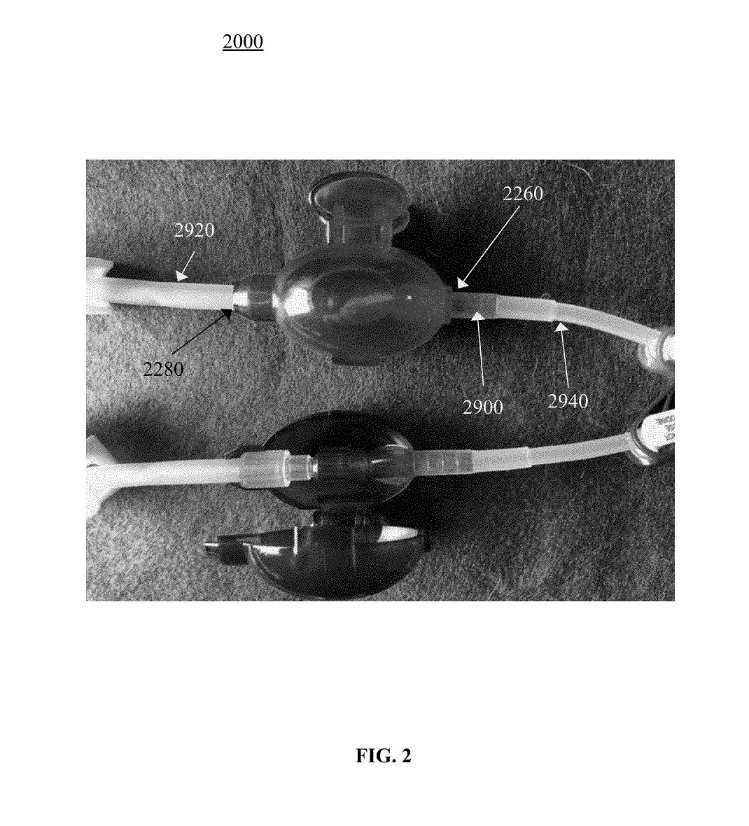

FIG. 2 is a perspective view of an exemplary embodiment of a system 2000, which illustrates exemplary dialysis lines coupled via exemplary couplings.

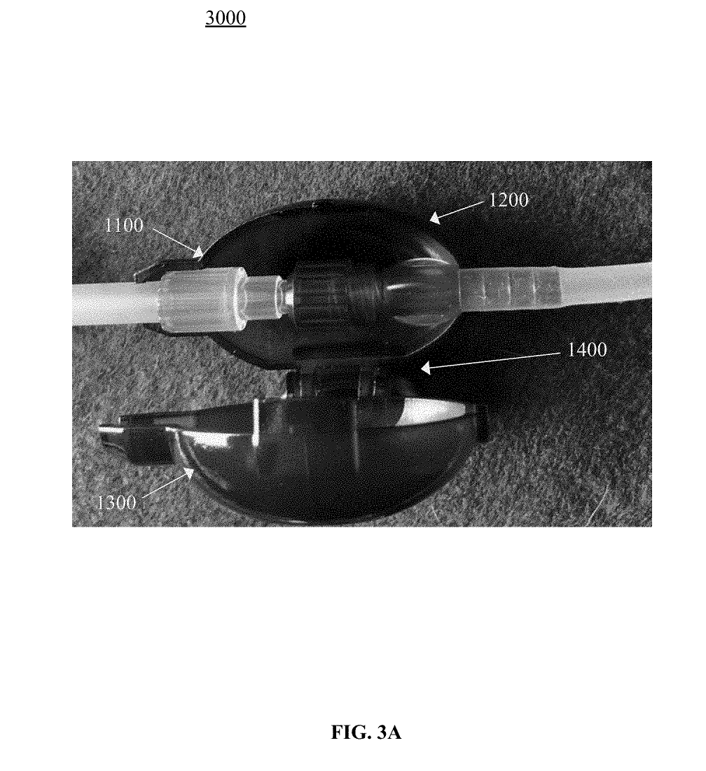

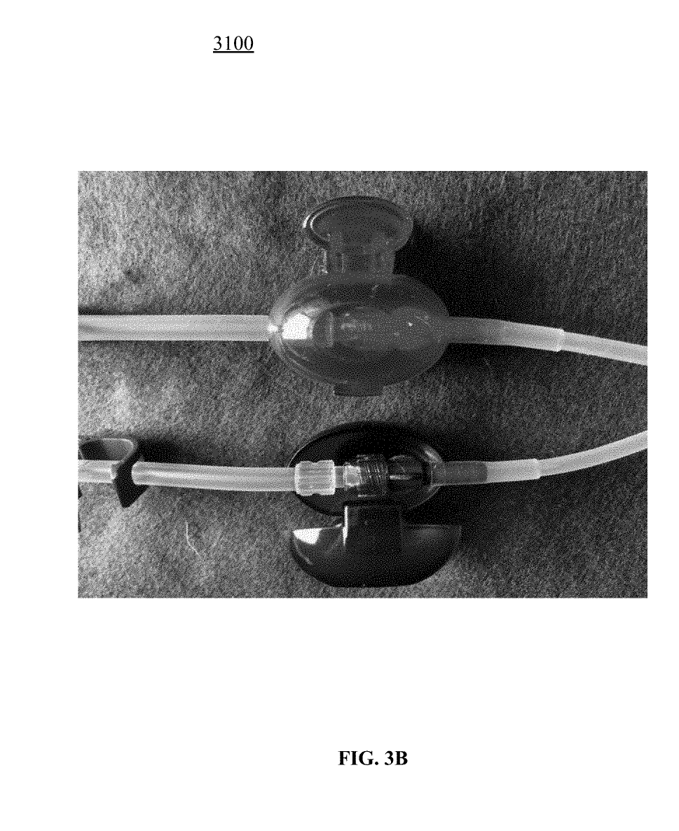

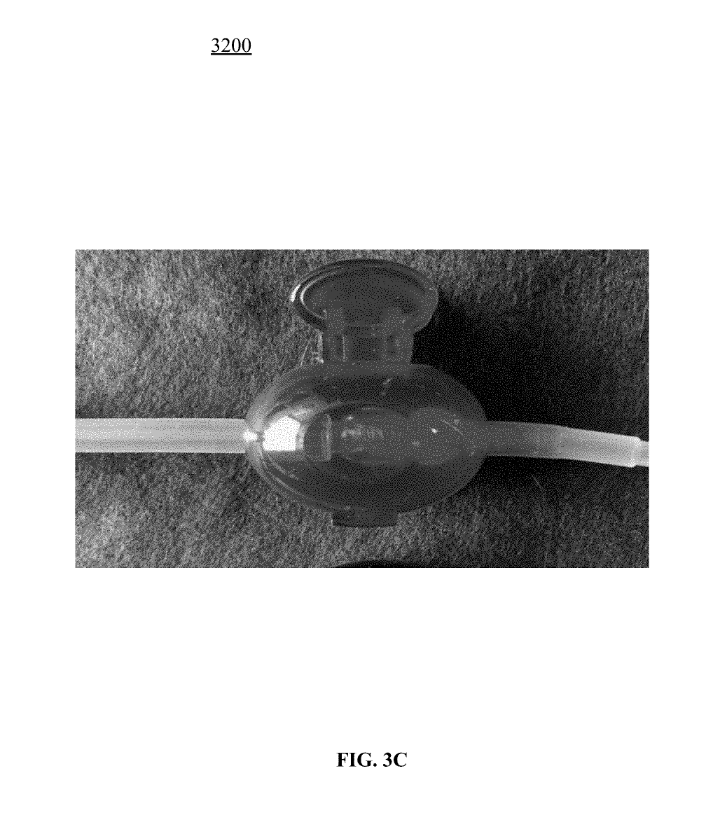

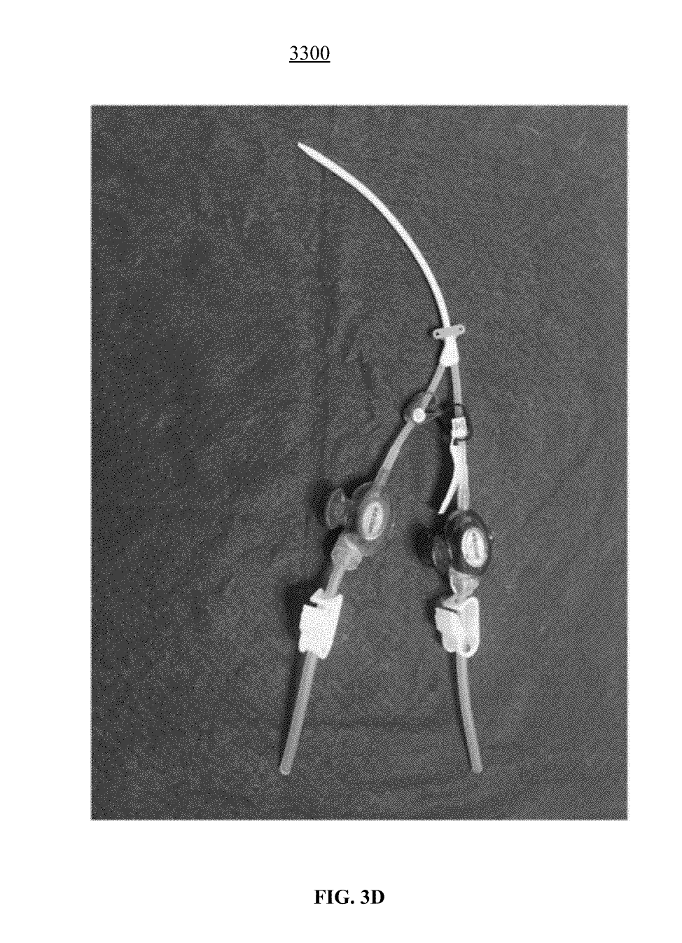

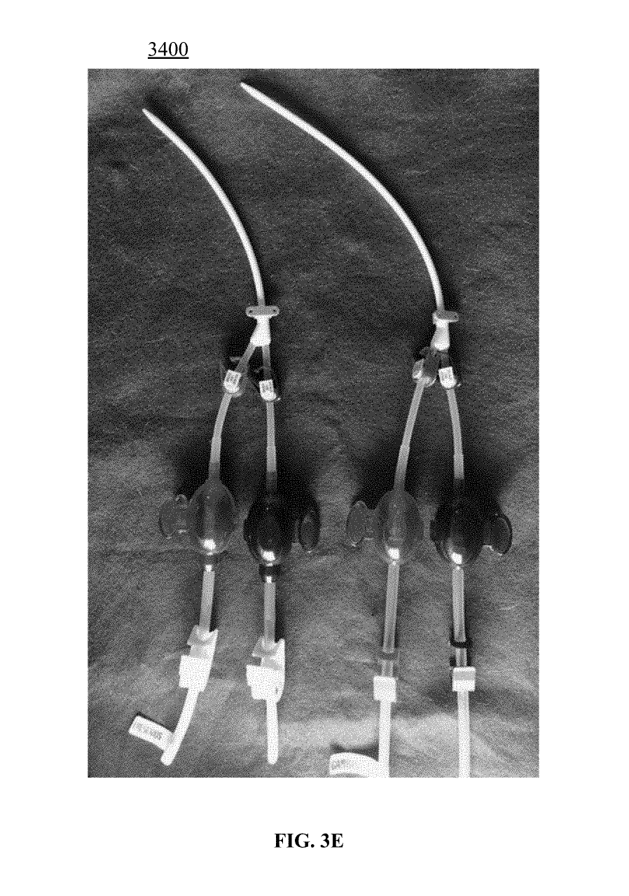

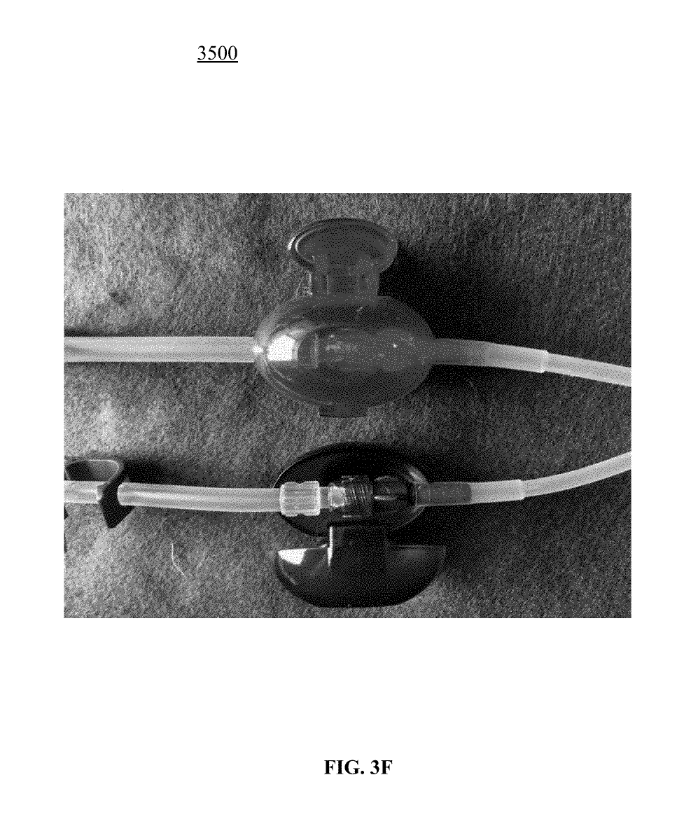

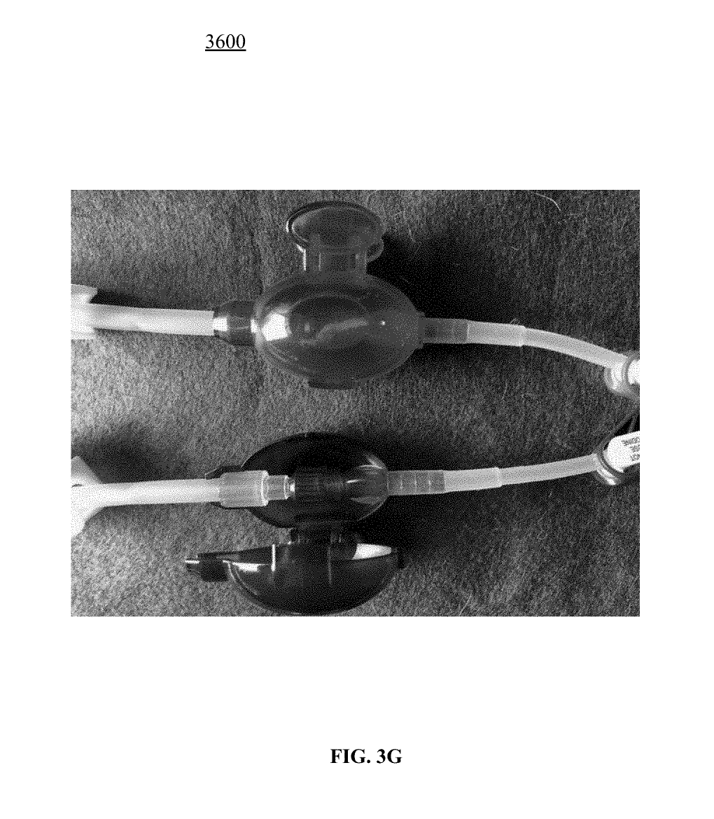

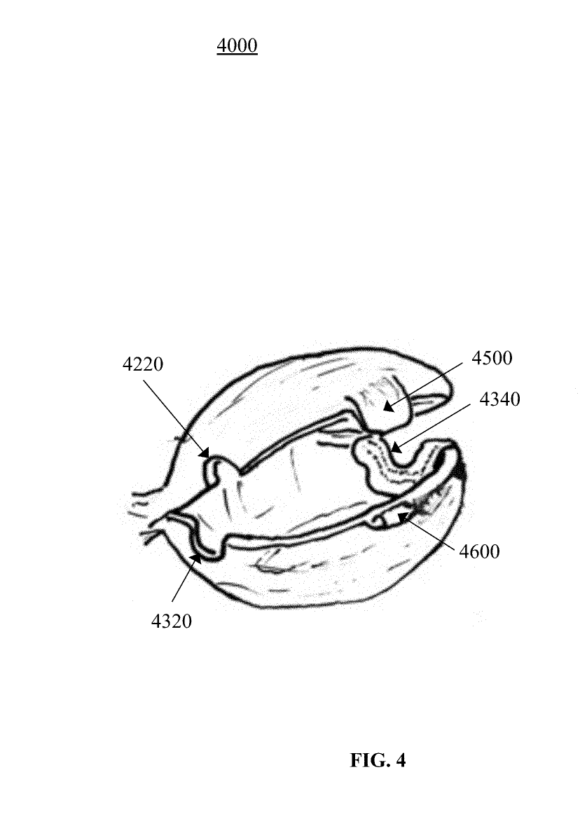

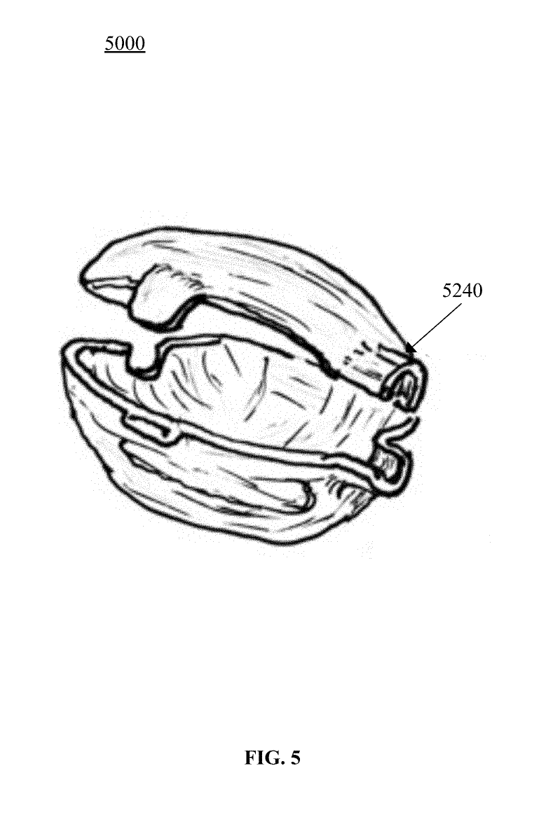

FIG. 3A is a perspective view of an exemplary embodiment of a system 3000, which illustrates exemplary dialysis lines coupled via an exemplary coupling. System 3000 comprises a shell 1100. In certain exemplary embodiments, shell 1100 is rigid. Shell 1100 comprises an upper portion 1200 and a lower portion 1300. Upper portion 1200 is coupled to lower portion 1300 via a hinge 1400. Upper portion 1200 defines a first upper slot (see first upper slot 4220 of FIG. 4) and a second upper slot (see second upper slot 5240 of FIG. 5). Lower portion 1300 defines a first lower slot (see first lower slot 4320 of FIG. 4) and a second lower slot (see second lower slot 4340 of FIG. 4). Upper portion 1200 comprises a clasp (see clasp 4500 of FIG. 4). Lower portion 1300 defines a clasp receptacle (see clasp receptacle 4600 of FIG. 4). When upper portion 1200 is closably coupled to lower portion 1300:
clasp 4500 is engaged with clasp receptacle 4600;
first upper slot 4220 and first lower slot 4320 define a first tubing aperture (see first tubing aperture 2260 of FIG. 2);
second upper slot 5240 and the second lower slot 4340 define a second tubing aperture (see second tubing aperture 2280 of FIG. 2); and
is constructed to substantially surround and enclose a coupled line (see coupled line 2900 of FIG. 2), which can be a catheter hub, a dialysis line, a hemodialysis connection, a peritoneal dialysis connection, and/or a blood line connection, etc.

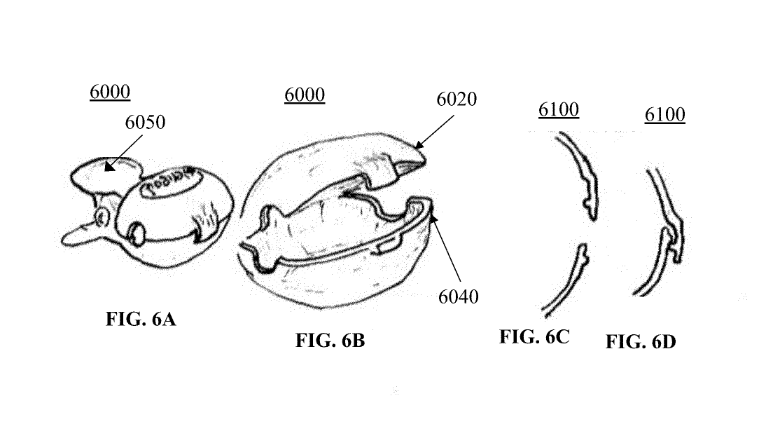

System 3000 can comprise one or more wings (see wings 6050 of FIG. 6A) on shell 1100 that are graspable by a human to separate upper portion 1200 from lower portion 1300.

Coupled line 2900, which can be a catheter hub, dialysis line, or blood line connection) can be Luer-locked, thereby securely coupling two tubing segments (see tubing segments 2920 and 2940 of FIG. 2).

Shell 1100 can be constructed to substantially surround a catheter connection. Shell 1100 can be constructed to engage both sides of a catheter. Shell 1100 can be constructed to substantially surround a dialysis line connection. Shell 1100 can be constructed to substantially surround a blood line connection.

Shell 1100 can be substantially transparent thereby permitting positive visualization of connector status during use.

FIG. 3B is a perspective view of an exemplary embodiment of a system 3100.

FIG. 3C is a perspective view of an exemplary embodiment of a system 3200.

FIG. 3D is a perspective view of an exemplary embodiment of a system 3300.

FIG. 3E is a perspective view of an exemplary embodiment of a system 3400.

FIG. 3F is a perspective view of an exemplary embodiment of a system 3500.

FIG. 3G is a perspective view of an exemplary embodiment of a system 3600.

FIG. 4 is a perspective view of an exemplary embodiment of a system 4000. Certain exemplary embodiments provide a firm but pliable silicon sleeve. Certain exemplary embodiments provide a suitable aperture diameter to provide traction on tubing surfaces.

FIG. 5 is a perspective view of an exemplary embodiment of a system 5000.

FIG. 6A is a perspective view of an exemplary embodiment of a system 6000, which can be a molded clip.

FIG. 6B is a perspective view of system 6000, which can comprise a molded upper projection 6020 and a molded lower projection 6040.

FIG. 6C is a clip cross section 6100 of system 6000 with the clip open.

FIG. 6D is a clip cross section 6100 of system 6000 with the clip shut.

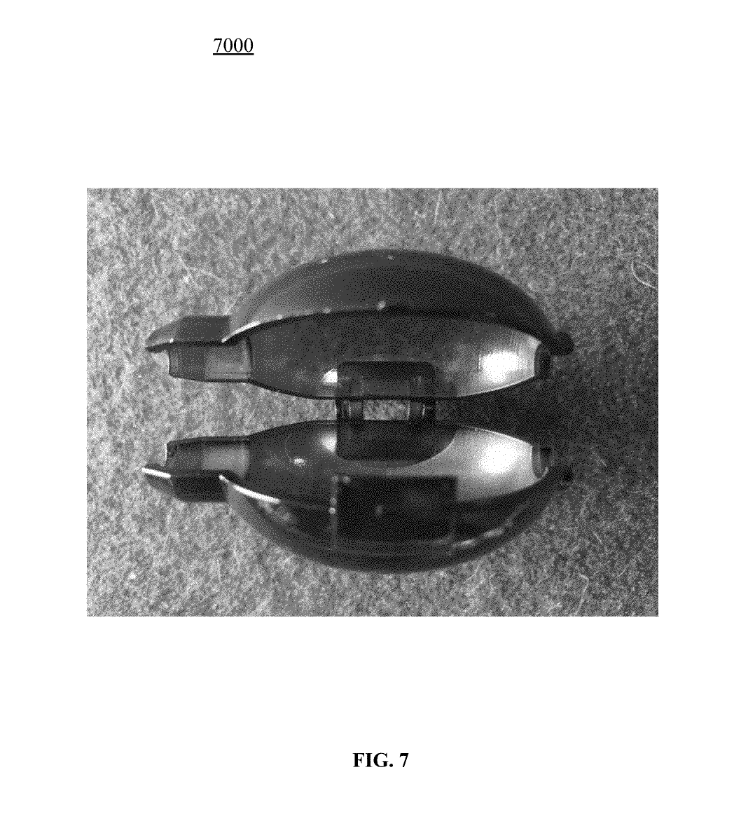

FIG. 7 is a perspective view of an exemplary embodiment of a system 7000.

### Definitions

When the following terms are used substantively herein, the accompanying definitions apply. These terms and definitions are presented without prejudice, and, consistent with the application, the right to redefine these terms during the prosecution of this application or any application claiming priority hereto is reserved. For the purpose of interpreting a claim of any patent that claims priority hereto, each definition (or redefined term if an original definition was amended during the prosecution of that patent), functions as a clear and unambiguous disavowal of the subject matter outside of that definition.
a—at least one.
activity—an action, act, step, and/or process or portion thereof
adapter—a device used to effect operative compatibility between different parts of one or more pieces of an apparatus or system.
and/or—either in conjunction with or in alternative to.
apparatus—an appliance or device for a particular purpose
associate—to join, connect together, and/or relate.
blood line—a tubing system that conveys blood.
can—is capable of, in at least some embodiments.
catheter hub—a coupling in a thin tube made from medical grade materials serving a broad range of functions.
clasp—a component that engages with a clasp receptacle to couple two pieces together.
clasp receptacle—a component that engages with a clasp to couple two pieces together.
closably—in a manner that substantially encloses something.
comprising—including but not limited to.
configure—to make suitable or fit for a specific use or situation.
connect—to join or fasten together.
constructed to—made suitable or fit for a specific use or situation.
convert—to transform, adapt, and/or change.
couple—to link in some fashion.
coupleable—capable of being joined, connected, and/or linked together.
define—to establish the outline, form, or structure of
device—a machine, manufacture, and/or collection thereof.
dialysis line—a tubing system that conveys blood to a dialyzer for removal of uric acid and urea.
enclose—to surround or close off substantially on all sides.
engage—to move into a position in which to components interlock.
generate—to create, produce, give rise to, and/or bring into existence.
hinge—a mechanical bearing that couples two solid objects, which allows only a limited angle of rotation between the two solid objects.
install—to connect or set in position and prepare for use.
lower—situated below something else in a system in a defined orientation.
may—is allowed and/or permitted to, in at least some embodiments.
method—a process, procedure, and/or collection of related activities for accomplishing something.
plurality—the state of being plural and/or more than one.
portion—a part of something.
predetermined—established in advance.
provide—to furnish, supply, give, and/or make available.
receive—to get as a signal, take, acquire, and/or obtain.
repeatedly—again and again; repetitively.
rigid—substantially inflexible.
segment—a piece or part.
set—a related plurality.
shell—an outer case that substantially surrounds something such that access to the surrounded something is restricted.
slot—an aperture having a curved edge and shaped substantially as half a circle.
store—to place, hold, and/or retain.
substantially—to a great extent or degree.
support—to bear the weight of, especially from below.
surround—to be around an object on substantially all sides.
system—a collection of mechanisms, devices, machines, articles of manufacture, processes, data, and/or instructions, the collection designed to perform one or more specific functions.
tubing—a hollow, flexible, cylindrical body constructed to convey fluids.
tubing aperture—a substantially round opening constructed to receive medical tubing.
transmit—to send, provide, furnish, and/or supply.
tubing—a system comprising one or more cylinders that constructed to transmit fluids via a channel interior to walls of the cylinders.
upper—situated above something else in a system in a defined orientation.
via—by way of and/or utilizing.

### NOTE

Still other substantially and specifically practical and useful embodiments will become readily apparent to those skilled in this art from reading the above-recited and/or herein-included detailed description and/or drawings of certain exemplary embodiments. It should be understood that numerous variations, modifications, and additional embodiments are possible, and accordingly, all such variations, modifications, and embodiments are to be regarded as being within the scope of this application.

Thus, regardless of the content of any portion (e.g., title, field, background, summary, description, abstract, drawing figure, etc.) of this application, unless clearly specified to the contrary, such as via explicit definition, assertion, or argument, with respect to any claim, whether of this application and/or any claim of any application claiming priority hereto, and whether originally presented or otherwise:
there is no requirement for the inclusion of any particular described or illustrated characteristic, function, activity, or element, any particular sequence of activities, or any particular interrelationship of elements;
no characteristic, function, activity, or element is “essential”;
any elements can be integrated, segregated, and/or duplicated;
any activity can be repeated, any activity can be performed by multiple entities, and/or any activity can be performed in multiple jurisdictions; and
any activity or element can be specifically excluded, the sequence of activities can vary, and/or the interrelationship of elements can vary.

Moreover, when any number or range is described herein, unless clearly stated otherwise, that number or range is approximate. When any range is described herein, unless clearly stated otherwise, that range includes all values therein and all subranges therein. For example, if a range of 1 to 10 is described, that range includes all values therebetween, such as for example, 1.1, 2.5, 3.335, 5, 6.179, 8.9999, etc., and includes all subranges therebetween, such as for example, 1 to 3.65, 2.8 to 8.14, 1.93 to 9, etc.

When any claim element is followed by a drawing element number, that drawing element number is exemplary and non-limiting on claim scope. No claim of this application is intended to invoke paragraph six of 35 USC 112 unless the precise phrase “means for” is followed by a gerund.

Any information in any material (e.g., a United States patent, United States patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such material is specifically not incorporated by reference herein.

Accordingly, every portion (e.g., title, field, background, summary, description, abstract, drawing figure, etc.) of this application, other than the claims themselves, is to be regarded as illustrative in nature, and not as restrictive, and the scope of subject matter protected by any patent that issues based on this application is defined only by the claims of that patent.

What is claimed is:

1. A system comprising:
a shell that comprises an upper portion and a lower portion, the upper portion coupled to the lower portion via a hinge, the upper portion defining a first upper slot and a second upper slot, the lower portion defining a first lower slot and a second lower slot, the upper portion comprising a clasp, the lower portion defining a clasp receptacle, wherein when the upper portion is closably coupled to the lower portion:
the clasp is engaged with the clasp receptacle;
the first upper slot and the first lower slot define a first tubing aperture, the first tubing aperture comprising a first lip and a second lip, the first lip and the second lip constructed to grip a first tubing fitting that is substantially surrounded by a first section of tubing;
the second upper slot and the second lower slot define a second tubing aperture, the second tubing aperture comprising a nipple, the nipple comprising a cylindrical section and a tapered section, the cylindrical section constructed to substantially surround a second tubing fitting, the tapered section constructed to substantially surround and grip a second section of tubing; and
is constructed to substantially surround and enclose a catheter hub, a dialysis line, or a blood line connection.

2. The system of claim 1, further comprising:
one or more wings on the shell that are graspable by a human to separate the upper portion from the lower portion, each of the one or more wings comprise a rectangular portion and an oval portion, wherein the rectangular portion of each of the one or more wings is coupled directly to the shell.

3. The system of claim 1, wherein:
the catheter hub, dialysis line, or blood line connection is Luer-locked, thereby coupling two tubing segments.

4. The system of claim 1, wherein:
the shell is constructed to substantially surround a catheter connection.

5. The system of claim 1, wherein:
the shell is constructed to securely engage both a top surface and a bottom surface of a fluid line connected to a hemodialysis catheter hub.

6. The system of claim 1, wherein:
the shell is constructed to substantially surround a dialysis line connection.

7. The system of claim 1, wherein:
the shell is constructed to substantially surround the blood line connection.

8. The system of claim 1, wherein:
the shell is rigid.

9. The system of claim 1, wherein:
the shell is substantially transparent thereby permitting positive visualization of connector status during use.

10. A method comprising:
causing two tubing segments to be coupled via a device, the tubing segments used on one of a catheter hub, dialysis line, or blood line connection, the device comprising:
a shell that comprises an upper portion and a lower portion, the upper portion coupled to the lower portion via a hinge, the upper portion defining a first upper slot and a second upper slot, the lower portion defining a first lower slot and a second lower slot, the upper portion comprising a clasp, the lower portion defining a clasp receptacle, wherein when the upper portion is closably coupled to the lower portion:
the clasp is engaged with the clasp receptacle;
the first upper slot and the first lower slot define a first tubing aperture, the first tubing aperture comprising a first lip and a second lip, the first lip and the second lip constructed to grip a tubing fitting that is substantially surrounded by a section of tubing; and
the second upper slot and the second lower slot define a second tubing aperture.

11. A method comprising:
causing two tubing segments to be coupled via a device, the tubing segments used on one of a catheter hub, dialysis line, or blood line connection, the device comprising:
a shell that comprises an upper portion and a lower portion, the upper portion coupled to the lower portion via a hinge, the upper portion defining a first upper slot and a second upper slot, the lower portion defining a first lower slot and a second lower slot, the upper portion comprising a clasp, the lower portion defining a clasp receptacle, wherein when the upper portion is closably coupled to the lower portion:
the clasp is engaged with the clasp receptacle;
the first upper slot and the first lower slot define a first tubing aperture; and
the second upper slot and the second lower slot define a second tubing aperture, the second tubing aperture comprising a nipple, the nipple comprising a cylindrical section and a tapered section, the cylindrical section constructed to substantially surround a tubing fitting, the tapered section constructed to substantially surround and grip a section of tubing.

12. The system of claim 1, wherein:
the shell substantially completely encircles/encloses the bloodline connection and therefore protects it against contamination with the clasp engaged during use.

13. The system of claim 1, wherein:
the a catheter hub, the dialysis line, or the blood line connection operates under pressure.
